SEQUENCE LISTING (10 sequences)

SEQ ID NO: 1 - DNA, 18 bp, Artificial Sequence
  Forward primer for VEGF
aactttctgctgtcttgg

SEQ ID NO: 2 - DNA, 18 bp, Artificial Sequence
  Reverse primer for VEGF
tttggtctgcattcacat

SEQ ID NO: 3 - DNA, 20 bp, Artificial Sequence
  Forward primer for aldolase A
gtcatcctcttccatgagac

SEQ ID NO: 4 - DNA, 20 bp, Artificial Sequence
  Reverse primer for aldolase A
aggtagatgtggtggtcact

SEQ ID NO: 5 - DNA, 20 bp, Artificial Sequence
  Forward primer for enolase I
aagaaactgaacgtcacaga

SEQ ID NO: 6 - DNA, 20 bp, Artificial Sequence
  Reverse primer for enolase I
gatcttcgatagacaccact

SEQ ID NO: 7 - DNA, 21 bp, Artificial Sequence
  Forward primer for HIF-1a
ccccagattcaggatcagaca

SEQ ID NO: 8 - DNA, 21 bp, Artificial Sequence
  Reverse primer for HIF-1a
ccatcatgttccatttttcgc

SEQ ID NO: 9 - DNA, 20 bp, Artificial Sequence
  Forward primer for beta-actin
aagagaggcatcctcaccct

SEQ ID NO: 10 - DNA, 20 bp, Artificial Sequence
  Reverse primer for beta-actin
atctcttgctcgaagtccag

(19) United States
(12) Patent Application Publication
(10) Pub. No.: US 2005/0096370 A1
(43) Pub. Date: May 5, 2005

(54) Method for inhibiting tumor angiogenesis and tumor growth

(75) Inventors: Jong-Wan Park, Seoul (KR); Yang-Sook Chun, Seoul (KR); Jinho Kim, Seoul (KR)

Correspondence Address: BEYER WEAVER & THOMAS LLP, P.O. BOX 70250, OAKLAND, CA, 94612-0250, US

(73) Assignee: Bizbiotech Co., Ltd.

(21) Appl. No.: 10/642,363

(22) Filed: Aug. 14, 2003

Related U.S. Application Data
(63) Continuation-in-part of application No. 10/407,136, filed on Apr. 7, 2003.

(51) Int. Cl. A61K 31/416

(52) U.S. Cl. 514/406

(57) ABSTRACT

The present invention provides methods and pharmaceutical compositions for inhibiting expressions of HIF-1 and HIF-1-regulated genes, angiogenesis, tumor growth, or tumor progression/metastasis comprising contacting the tumor cells or tissue with a composition comprising 3-(5′-hydroxymethyl-2′-furyl)-1-benzylindazole, or a novel derivative thereof.

### CROSS REFERENCE TO RELATED APPLICATIONS

This application claims priority to U.S. patent application Ser. No. 10/407,136, filed Apr. 7, 2003 (Attorney Docket No. BIZBP004), which application is incorporated herein by reference in its entirety for all purposes.

### FIELD OF THE INVENTION

The present invention relates to methods and pharmaceutical compositions for inhibiting tumor angiogenesis and tumor growth. We have found that YC-1,3-(5′-hydroxymethyl-2′-furyl)-1-benzylindazole and novel derivatives thereof, inhibit hypoxic induction of hypoxia-inducible factor 1α (HIF-1α) transcription factor and the expression of a key angiogenic factor, vascular endothelial growth factor (VEGF) in various human cancer cell-lines in vitro and in vivo. Moreover, the compounds of the invention halt the growth of the xenografted tumors originating from these cancer cell-lines. Therefore, we provide methods and pharmaceutical compositions of using YC-1 and novel derivatives thereof for the purpose of treatment of developed tumors and prevention of metastasis and carcinogenesis in animals.

### BACKGROUND OF THE INVENTION

Hypoxia, a reduction in tissue oxygen levels below physiologic levels, 20 commonly develops within solid tumors because tumor cell proliferation is greater than the rate of blood vessel formation. Thus, the increase in tumor mass results in aberrant vasculature formation, which compromises the blood supply (Hockel et al., J Natl Cancer Inst 2001 93:266-276). Tumor hypoxia is one stimulus that leads to the increased expression of vascular endothelial growth factor (VEGF) and stimulates 25 angiogenesis, which is essential for meeting the metabolic requirements of tumor growth (Dachs et al., Eur J Cancer 2000 36:1649-1660). In addition, hypoxia contributes to tumor progression to a more malignant phenotype because cells surviving under hypoxic conditions often become resistant to radiotherapy and chemotherapy (Brown, J. M. Cancer Res 1999 59:5863-5870). Thus, factors that regulate hypoxic events may be good targets for anticancer therapy.

One such target is hypoxia-inducible factor 1 (HIF-1). HIF-1 is a key transcription factor that regulates the blood supply through the expression of vascular endothelial growth factor (VEGF) (Forsythe et al., Mol Cell Biol 1996 16:4604-4613). The biologic activity of HIF-1, a heterodimer composed of HIF-1α and HIF-1β (Wang et al., J Biol Chem 1995 270:1230-1237), depends on the amount of HIF-1α, which is tightly regulated by oxygen tension. Under normoxic conditions, HIF-1α protein is unstable. The instability is regulated, in part, by the binding to the von Hippel-Lindau tumor suppressor protein (pVHL) (Maxwell et al., Nature 1999 399:271-275). This binding occurs after the hydroxylation of the two HIF-1α proline residues by HIF-propyl hydroxylases (Jaakkola et al., Science 2001 292:468-472; Ivan et al., Science 2001 292:464-468; Masson et al., EMBO J. 2001 20:5197-5206). The von Hippel-Lindau protein is one of the components of the multiprotein ubiquitin-E3-ligase complex, which mediates the ubiquitylation of HIF-1α, targeting it for proteasomal proteolysis (Huang et al., Proc Natl Acad Sci USA 1998 95:7987-7992). However, under hypoxic conditions, proline hydroxylation is inhibited, binding between HIF-1 and the von Hippel-Lindau protein is eliminated and HIF-1 a becomes stable.

A growing body of evidence indicates that HIF-1 contributes to tumor progression and metastasis. In human tumors, HIF-1α is overexpressed as a result of intratumoral hypoxia and genetic alterations affecting key oncogenes (HER2, FRAP, HRAS, and CSRC and tumor suppressor genes (VHL, PTEN, and p53) (Semenza, G. L. Trends Mol Med 2002 8:S62-S67). Immunohistochemical analyses show that HIF 1α is present at higher levels in human tumors than in normal tissues (Zhong et al., Cancer Res 1999 59:5830-5835). Moreover, the expression of HIF-1α in biopsy specimens from various solid tumors has been associated with tumor aggressiveness, vascularity, treatment failure, and mortality (Birner et al., Cancer Res 2000 60:4693 4696). In addition, tumor growth and angiogenesis in xenografted tumors also depends on HIF-1 activity and on the expression level of HIF-1α (Maxwell et al., Proc Natl Acad Sci USA 1997 94:8104-8109). Therefore, because HIF-1 activity appears central to tumor progression and metastasis, inhibition of HIF-1 activity must be an appropriate anticancer target. While searching for an antiangiogenic agent that would inhibit HIF-1 activity, we identified a novel pharmacologic action of YC-1. YC-1, 3-(5′-hydroxymethyl-2′ furyl)-1-benzylindazole, inhibits platelet aggregation and vascular contraction by activating soluble guanylyl cyclase, and was originally developed as a potential therapeutic agent for circulation disorders (Teng et al., Eur J Pharmacol 1997 320:161 166; Galle et al., Br J Pharmacol 1999 127:195-203).

Recently, we have found a new biological action of YC-1 to block the hypoxic activation of HIF-1 (Chun et al., Biochem Pharmacol 2001 61:947-954). YC-1 reduced HIF-1α expression at the post transcriptional level and inhibited its transcriptional activity in hepatoma cells cultured under hypoxic conditions. These effects of YC-1 are likely to be linked with the oxygen-sensing pathway, and not with the activation of soluble guanylyl cyclase.

Meanwhile, U.S. Patent Application Publication No. 2002/0040059 A1 discloses methods and formulations for inhibiting and preventing a malignant cell phenotype using a low dose of nitric oxide (NO) mimetic. In the detailed description of this US application, it is described that NO mimetic includes any compounds which act as the NO pathway mimetic, however, preferably, the NO mimetic does not encompass a compound which activates directly either particulate- or soluble guanylyl cyclase, i.e., YC-1, in some embodiments. Practically, the antiangiogenic and antitumorigenic effects of YC-1 have been neither studied nor identified in this US application.

In the present invention, we tested whether YC-1 could target HIF-1 and inhibit tumor angiogenesis in vivo. We confirmed the inhibitory effects of YC-1 on the expression of HIF-1α and on the induction of VEGF, aldolase A, and enolase I in cancer cells cultured under hypoxic conditions. In vivo, treatment with YC-1 halted the growth of xenografted tumors originating from hepatoma, stomach carcinoma, renal carcinoma, cervical carcinoma, and neuroblastoma cells. Tumors from YC-1-treated mice showed fewer blood vessels and reduced expression of HIF-1α protein and HIF-1-regulated genes than tumors from vehicle-treated mice. These results support that YC-1 is an inhibitor of HIF-1 that halts tumor growth by blocking tumor angiogenesis and tumor adaptation to hypoxia.

### SUMMARY OF THE INVENTION

The present invention features the antiangiogenic and anticancer effects of YC-1 and novel derivatives thereof through the inhibition of HIF-1α.

The novel derivatives of YC-1 comprising compounds of Formula 1: 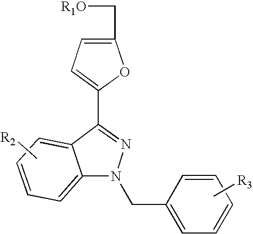
are provided wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
   including single isomers, mixtures of isomers, and pharmaceutically acceptable solvates and salts thereof. The invention also provides methods and pharmaceutical compositions for combining YC-1 or compounds of Formula 1 with other anticancer agents.

### BRIEF DESCRIPTION OF THE DRAWINGS

FIG. 1 is an immunoblot showing the effects of YC-1 on the expression of HIF-1 α and hypoxia-inducible genes in Hep3B hepatoma cells. Hep3B cells were treated with the indicated concentrations of YC-1 for 5 minutes before being cultured for 4 hours under normoxic (N, 20%02 V/V) or hypoxic (H, 1% 02 V/V) conditions. Expressions of HIF-1α and β-actin proteins were analyzed by immunoblotting with a rabbit anti-HIF-1α antibody and a rabbit anti-p-actin antibody, respectively. Proteins were visualized by enhanced chemiluminescence.

FIG. 2 is an autoradiograph showing mRNA levels of VEGF, aldolase A, enolase 1, HIF-1α, and β-actin. Total RNA was isolated from Hep3B cells that had been treated with the indicated concentrations of YC-1 and cultured under normoxic (N) or hypoxic (H) conditions for 16 hours. mRNA expression was analyzed by semiquantitative reverse transcription-polymerase chain reaction.

FIG. 3 is a histogram showing the amount of VEGF protein. VEGF protein in conditioned media from Hep3B cells that had been treated with the indicated concentrations of YC-1 and cultured under normoxic (N) or hypoxic (H) conditions for 24 hours was measured using an ELISA kit. The VEGF concentrations were quantified by comparison with a series of VEGF standard samples included in the assay kit. VEGF level in each experiment was measured twice. Bars represent the mean of four separate experiments with the 95% confidence interval. * denotes statistical significance compared to control supernatants from cells cultured under normoxic conditions (P<0.001); # denotes statistical significance compared to control supernatants from cells cultured under hypoxic conditions (P<0.00 1).

FIG. 4 is an immunoblot showing the effect of YC-1 on the expression of HIF-1α and VEGF in cancer cells of different origin. NCI-H87 gastric carcinoma, SiHa cervical carcinoma, SK-N-MC neuroblastoma, and Caki-1 renal carcinoma cells were treated with the indicated concentrations of YC-I for 5 minutes before being cultured under normoxic (N, 20%02 v/v) or hypoxic (H, 1% O₂ v/v) conditions for 4 hours. Levels of HIF-1α and β-actin proteins were analyzed by immunoblot analysis using a rabbit anti-HIF-1α antibody or a rabbit anti-p-actin antibody. Proteins were visualized by enhanced chemiluminescence.

FIG. 5 is an autoradiograph showing mRNA levels of VEGF and β-actin in cancer cells of different origin. NCI-H87 gastric carcinoma, SiHa cervical carcinoma, SK-N-MC neuroblastoma, and Caki-1 renal carcinoma cells were treated with the indicated concentrations of YC-1 for 5 minutes before being cultured under normoxic (N, 20%02 v/v) or hypoxic (H, 1%02 v/v) conditions for 16 hours. mRNA expression was analyzed by semi-quantitative reverse transcription-polymerase chain reaction.

FIG. 6 reveals growth curves of human tumors grafted in the flanks of nude mice. Male nude mice were injected subcutaneously in the flank with 5×10⁶ viable Hep3B hepatoma (A), NCI-H87 gastric carcinoma (B), SiHa cervical carcinoma (C), SK-N-MC neuroblastoma (E), or Caki-I renal carcinoma (F) cells. After the tumors reached 100 to 150 mm³ in size (indicated by long arrows), mice received an intraperitoneal injection of YC-1 (30 μg/g) or vehicle (DMSO) daily for 2 weeks. Tumor size was measured over time. A) Beginning 2 days after the injection of the Hep3B cells, some mice received injections of YC-1 daily for 2 weeks (indicated by a short arrow). Vehicle=solid circles, YC-1 (established tumors)=open circles, YC-1′ (treatment before established tumors)=open triangles. Each data point represents mean (n=12 for control; n=6 for YC-1; n=7 for YC-1′) and 95% confidence interval. Differences between tumor sizes in the vehicle- and YC-1-treated groups for mice with Hep3B tumors were compared using ANOVA and Duncan's multiple range tests. * denotes P<0.001 relative to the control. Differences between tumor sizes in the vehicle and YC-1-treated groups for mice with other tumors (B-E) were compared using a Mann-Whitney U test. Each number beneath the error bar represents the P value of the difference relative to the control.

FIG. 7 is a picture showing histopathology for Hep3B hepatoma tumors grown in nude mice. Male nude mice were injected subcutaneously in the flank with 5×10⁶ viable Hep3B cells. After the tumors reached 100 to 150 mrn˜ in size, mice received an intraperitoneal injection of YC-1 (30 μg/g) or vehicle (DMSO) daily for 2 weeks. After the last treatment, the mice were euthanized, the tumors removed, fixed with formalin, and embedded in paraffin. Tumor sections were cut from the paraffin blocks and stained with hernatoxylin and eosin. v, vessel; a, acinus. Scale bar=50 μm.

FIG. 8 is a picture showing vascular distribution in Hep3B hepatoma tumors grown in nude mice. Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed, fixed with formalin, and embedded in paraffin. Tumor sections were cut from the paraffin blocks and processed for immunohistochemical staining to detect endothelial cells with an anti-CD31 antibody. The immunostained sections were developed using the avidinbiotin-horseradish peroxidase method with diaminobenzidine as the chromagen. The sections were lightly counterstained with hematoxylin. Arrows indicate CD31-positive vessels. Scale bar=50 μm.

FIG. 9 is a picture showing expression and distribution of HIF-1α in Hep3B hepatoma tumors grown in nude mice. Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed, fixed with formalin, and embedded in paraffin. Tumor sections were cut from the paraffin blocks and processed for immunohistochemical staining to detect HIF-1α with an anti-HIF-1α antibody. The immunostained sections were developed using the avidin-biotin-horseradish peroxidase method with diaminobenzidine as the chromagen. The sections were lightly counterstained with hematoxylin. Arrows indicate HIF-1α positive cells. nu, nuclear staining; pn, perinuclear staining. Scale bar=50 μm.

FIG. 10 is a histogram showing HIF-1α expression detected by immunohistochemistry in human cancer xenografts derived from Hep3B hepatoma, NCI-H87 gastric carcinoma, SiHa cervical carcinoma, SK-N-MC neuroblastoma, or Caki-1 renal carcinoma cells. Male nude mice were injected subcutaneously in the flank with 5×10⁶ viable tumor cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed, fixed with formalin, embedded in paraffin and processed for hinmunohistochemistry to detect HIF-1α-positive cells. Two sections per xenograft (5-10 fields per section) were examined for histologic assessment (control and YC-1: n=24 and 12 for Hep3B, n=14 and 12 for NCI-H87, n=10 and 10 for SiHa, n=10 and 10 for SK-N-MC, n=12 and 10 for Caki-I). Each bar represents the mean with lower or upper 95% confidence interval. Differences between treatment groups were compared using a Mann-Whitney U test. Each number over the error bar represents the P value of the difference relative to the control value.

FIG. 11 is a histogram showing vascular density detected by immunohistochemistry in human cancer xenografts derived from Hep3B hepatoma, NCI-H87 gastric carcinoma, SiHa cervical carcinoma, SK-N-MC neuroblastoma, or Caki-1 renal carcinoma cells. Male nude mice were injected subcutaneously in the flank with 5×10⁶ viable tumor cells. After the tumors reached 100 to 150 in size, mice received an intraperitoneal injection of YC-1 (30 mg/g) or vehicle (DMSO) daily for 2 weeks. After the last treatment, the mice were euthanized, the tumors removed, fixed with formalin, embedded in paraffin and processed for immunohistochemistry to detect CD31-positive cells. Two sections per xenograft (5-10 fields per section) were examined for histologic assessment (control and YC-1: n=24 and 12 for Hep3B, n=14 and 12 for NCI-H87, n=10 and 10 for SiHa, n=10 and 10 for SK-N-MC, n=12 and 10 for Caki-I). Each bar represents the mean with lower or upper 95% confidence interval. Differences between treatment groups were compared using a Mann-Whitney U test. Each number over the error bar represents the P value of the difference relative to the control value.

FIG. 12 is an immunoblot showing the effects of YC-1 on the expression of HIF-1α and VEGF in Hep3B hepatoma cell xenografts. Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed and lysates prepared for immunoblotting. Tumor lysates from vehicle-treated mice (C) and from YC-1-treated (YC-1) mice were assessed by immunoblotting for HIF-1α, VEGF, and β-actin protein levels.

FIG. 13 is an autoradiograph showing the effects of YC-1 on the expression of hypoxia-inducible genes in Hep3B hepatoma cell xenografts. Male nude mice were injected subcutaneously in the flank with 5×10⁶ viable Hep3B cells. After the tumors reached 100 to 150 mm³ in size, mice received an intraperitoneal injection of YC-1 (30 μ/g) or vehicle (DMSO) daily for 2 weeks. After the last treatment, the mice were euthanized, the tumors removed and lysates prepared for mRNA analysis. The mRNA levels of VEGF, aldolase A, enolase 1, and β-actin were measured by semi-quantitative RT-PCR. The quality of the extracted RNAs was verified by identifying the 18S ribosomal RNA (rRNA) on a 1% denaturing agarose gel.

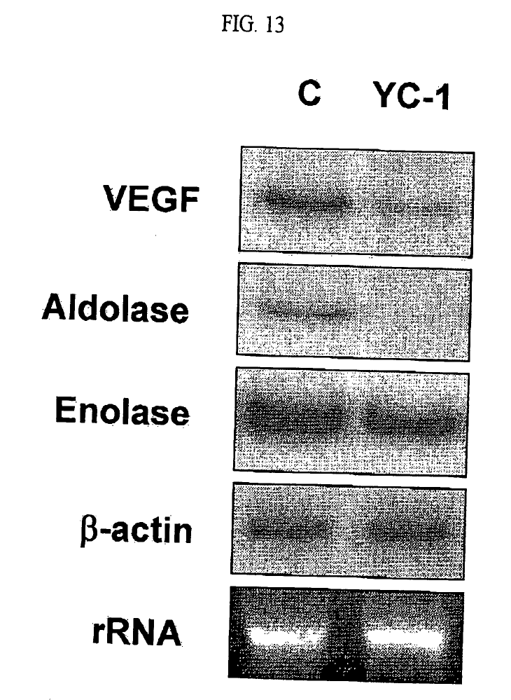

FIG. 14 is a graph showing the effect of YC-1 on natural killer (NK) cell activity. A) Splenic lymphocytes (6.25×10⁴ to 5×10⁵) isolated from male nude mice, were incubated with YC-1 at various concentrations for 24 hours. The lymphocytes were then incubated at the indicated effectontarget cell (E:T) ratios with ⁵¹Cr-labeled YAC-1 cells (1×10⁴). After 4 hours, the amount of radioactivity in the culture supernatants was measured with a gamma counter. B) Male nude mice (n=4 per group) received a daily intraperitoneal injection of vehicle (DMSO) or YC-1 (30 μg/g) for 2 weeks. Splenic lymphocytes were then isolated and tested for NK cell activity. The result is expressed as the mean of four separate experiments with 95% confidence intervals.

FIG. 15 is a graph showing the growth curves of human PC-3 prostate tumors grafted in the flanks of nude mice. The procedure is that described in connection with FIG. 6. and Example 5.

FIG. 16 is a graph showing the growth curves of human Hep3B hepatoma cells grafted in the flanks of nude mice. The comparison is among the growth inhibition respectively caused by intraperitoneal injection of YC-1 and YC-1 mannose derivative at two dosages. The procedure is that described in Examples 9 and 10.

FIG. 17 is a scheme showing the synthesis of YC-1-sugar derivatives starting with a sugar, mannose (201), and YC-1.

### DETAILED DESCRIPTION OF THE INVENTION

The present invention is based in part on the discovery that YC-1 and novel derivatives thereof can substantially inhibit the expressions of HIF-1α and the HIF-1-regulated genes in vitro and in vivo. It is also based on the discovery that YC-1 and novel derivatives thereof can have anticancer effect in vivo by blocking tumor angiogenesis essential for tumor growth and metastasis.

Accordingly, one aspect of the present invention provides a method of inhibiting HIF-1α expression in tumor cells or tissues, comprising contacting the tumor cells or tissues with a composition comprising YC-1 or novel derivatives thereof at an effective amount for inhibiting HIF-1α.

Another aspect of the present invention provides a method of inhibiting HIF-1-regulated gene expression in tumor cells or tissues, comprising contacting the tumor cells or tissues with a composition comprising YC-1 or novel derivative thereof at an effective amount for inhibiting HIF-1-regulated gene expression.

The other aspect of the present invention provides a method of inhibiting angiogenesis in tumor cells or tissues, comprising contacting the tumor cells or tissues with a composition comprising YC-1 or novel derivatives thereof at an effective amount for inhibiting angiogenesis.

A further aspect of the present invention provides a method of inhibiting tumor growth in animal tissues, comprising contacting the animal tissues with a composition comprising YC-1 or novel derivatives thereof at an effective amount for inhibiting tumor growth.

Yet another aspect of the present invention provides a method of inhibiting tumor progression and metastasis in animal tissues, comprising contacting the animal tissues with a composition comprising YC-1 or novel deratives thereof at an effective amount for inhibiting tumor progression and metastasis.

A further aspect of the present invention provides a method of treating a HIF-1-mediated disorder or condition in a mammal, comprising administering to the mammal a composition including a therapeutically effective amount of YC-1 or a novel derivative thereof.

The novel derivatives of YC-1 comprising compounds of Formula 1 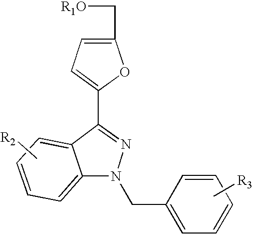
are provided wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
   including single isomers, mixtures of isomers, and pharmaceutically acceptable solvates and salts thereof.

In the present invention, the amount or dosage range of active compounds employed is one that effectively inhibits expressions of HIF-1α and HIF-1-related genes, angiogenesis, tumor growth, and/or tumor progression and metastasis. It is preferred that the effective amount of active compound is 2˜100 μM for inhibiting expressions of HIF-1α and HIF-1-related genes in the cell culture system and 5˜30 μg/g for inhibiting tumor angiogenesis and growth in vivo.

As used in the present specification, the following words and phrases are generally intended to have the meanings as set forth below, except to the extent that the context in which they are used indicates otherwise.

“Alkyl” is intended to include linear, branched, or cyclic hydrocarbon structures and combinations thereof. Lower alkyl refers to alkyl groups of from 1 to 5 carbon atoms. Examples of lower alkyl groups include methyl, ethyl, propyl, isopropyl, butyl, s- and t-butyl and the like. Preferred alkyl groups are those of C₂₀ or below. More preferred alkyl groups are those of C₁₃ or below. Still more preferred alkyl groups are those of C₆ and below. Cycloalkyl is a subset of alkyl and includes cyclic hydrocarbon groups of from 3 to 13 carbon atoms. Examples of cycloalkyl groups include c-propyl, c-butyl, c-pentyl, norbornyl, adamantyl and the like. In this application, alkyl refers to alkanyl, alkenyl and alkynyl residues; it is intended to include cyclohexylmethyl, vinyl, allyl, isoprenyl and the like. Alkylene is another subset of alkyl, referring to the same residues as alkyl, but having two points of attachment. Examples of alkylene include ethylene (—CH₂CH₂—), propylene (—CH₂CH₂CH₂—), dimethylpropylene (—CH₂C(CH₃) ₂CH₂—) and cyclohexylpropylene (—CH₂CH₂CH(C₆H₁₃)—). When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl.

The term “alkoxy” or “alkoxyl” refers to the group —O-alkyl, preferably including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

The term “substituted alkoxy” refers to the group —O-(substituted alkyl). One preferred substituted alkoxy group is “polyalkoxy” or —O-(optionally substituted alkylene)-(optionally substituted alkoxy), and includes groups such as —OCH₂CH₂OCH₃, and glycol ethers such as polyethyleneglycol and —O(CH₂CH₂O)ₓCH₃, where x is an integer of about 2-20, preferably about 2-10, and more preferably about 2-5. Another preferred substituted alkoxy group is hydroxyalkoxy or —OCH₂(CH₂)_{y}OH, where y is an integer of about 1-10, preferably about 1-4. “Acyl” refers to groups of from 1 to 10 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. “Lower-acyl” refers to groups containing 1 to 4 carbons and “acyloxy” refers to the group O-acyl.

The term “amino” refers to the group —NH₂. The term “substituted amino” refers to the group —NHR or —NRR where each R is independently selected from the group: optionally substituted alkyl, optionally substituted alkoxy, optionally substituted amino, optionally substituted aryl, optionally substituted heteroaryl, optionally substituted heterocyclyl, acyl, alkoxycarbonyl, sulfanyl, sulfinyl and sulfonyl, e.g., diethylamino, methylsulfonylamino, furanyl-oxy-sulfonamino.

“Aryl” and “heteroaryl” mean a 5-, 6- or 7-membered aromatic or heteroaromatic ring containing 0-4 heteroatoms selected from O, N or S; a bicyclic 9- or 10-membered aromatic or heteroaromatic ring system containing 0-4 (or more) heteroatoms selected from O, N or S; or a tricyclic 12- to 14-membered aromatic or heteroaromatic ring system containing 0-4 (or more) heteroatoms selected from O, N or S. The aromatic 6- to 14-membered aromatic carbocyclic rings include, e.g., phenyl, naphthalene, indane, tetralin, and fluorene and the 5- to 10-membered aromatic heterocyclic rings include, e.g., imidazole, oxazole, isoxazole, oxadiazole, pyridine, indole, thiophene, benzopyranone, thiazole, furan, benzimidazole, quinoline, isoquinoline, quinoxaline, pyrimidine, pyrazine, tetrazole and pyrazole.

“Aralkyl” refers to a residue in which an aryl moiety is attached to the parent structure via an alkyl residue. Examples include benzyl, phenethyl, phenylvinyl, phenylallyl and the like.

“Heteroaralkyl” refers to a residue in which a heteroaryl moiety is attached to the parent structure via an alkyl residue. Examples include furanylmethyl, pyridinylmethyl, pyrimidinylethyl and the like.

“Halogen” or “halo” refers to fluorine, chlorine, bromine or iodine. Fluorine, chlorine and bromine are preferred. Dihaloaryl, dihaloalkyl, trihaloaryl etc. refer to aryl and alkyl substituted with a plurality of halogens, but not necessarily a plurality of the same halogen; thus 4-chloro-3-fluorophenyl is within the scope of dihaloaryl.

“Heterocycle” means a cycloalkyl or aryl residue in which one to four of the carbons is replaced by a heteroatom such as oxygen, nitrogen or sulfur. Examples of heterocycles that fall within the scope of the invention include imidazoline, pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, oxadiazole, dioxane, tetrahydrofuran and the like. “N-heterocyclyl” refers to a nitrogen-containing heterocycle as a substituent residue. The term heterocyclyl encompasses heteroaryl, which is a subset of heterocyclyl. Examples of N-heterocyclyl residues include 4-morpholinyl, 4-thiomorpholinyl, 1-piperidinyl, 1-pyrrolidinyl, 3-thiazolidinyl, piperazinyl and 4-(3,4-dihydrobenzoxazinyl). Examples of substituted heterocyclyl include 4-methyl-1-piperazinyl and 4-benzyl-1-piperidinyl.

“Substituted-” alkyl, aryl, heteroaryl and heterocyclyl refer respectively to alkyl, aryl, heteroaryl and heterocyclyl wherein one or more (up to about 5, preferably up to about 3) hydrogen atoms are replaced by a substituent independently selected from the group: optionally substituted alkyl (e.g., fluoroalkyl), optionally substituted alkoxy, alkylenedioxy (e.g. methylenedioxy), optionally substituted amino (e.g., alkylamino and dialkylamino), optionally substituted amidino, optionally substituted aryl (e.g., phenyl), optionally substituted aralkyl (e.g., benzyl), optionally substituted aryloxy (e.g., phenoxy), optionally substituted aralkoxy (e.g., benzyloxy), carboxy (—COOH), carboalkoxy (i.e., acyloxy or —OOCR), carboxyalkyl (i.e., esters or —COOR), carboxamido, aminocarbonyl, benzyloxycarbonylamino (CBZ-amino), cyano, carbonyl, halogen, hydroxy, optionally substituted heteroaryl, optionally substituted heteroaralkyl, optionally substituted heteroaryloxy, optionally substituted heteroaralkoxy, nitro, sulfanyl, sulfinyl, sulfonyl, and thio.

The term “sulfanyl” refers to the groups: —S-(optionally substituted alkyl), —S-(optionally substituted aryl), —S-(optionally substituted heteroaryl), and —S-(optionally substituted heterocyclyl).

The term “sulfinyl” refers to the groups: —S(O)—H, —S(O)-(optionally substituted alkyl), —S(O)-(optionally substituted amino), —S(O)-(optionally substituted aryl), —S(O)-(optionally substituted heteroaryl), and —S(O)-(optionally substituted heterocyclyl).

The term “sulfonyl” refers to the groups: —S(O₂)—H, —S(O₂)-(optionally substituted alkyl), —S(O₂)-(optionally substituted amino), —S(O₂)-(optionally substituted aryl), —S(O₂)-(optionally substituted heteroaryl), —S(O₂)-(optionally substituted heterocyclyl), —S(O₂)-(optionally substituted alkoxy), —S(O₂)-optionally substituted aryloxy), —S(O₂)-(optionally substituted heteroaryloxy), and —S(O₂)-(optionally substituted heterocyclyloxy).

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl,” as defined below. It will be understood by those skilled in the art with respect to any group containing one or more substituents that such groups are not intended to introduce any substitution or substitution patterns that are sterically impractical, synthetically non-feasible and/or inherently unstable.

A “polyol” is a substance with multiple hydroxyl groups, and includes sugars (reducing and nonreducing sugars), sugar alcohols and sugar acids. Preferred polyols herein have a molecular weight which is less than about 600 kD (e.g. in the range from about 120 to about 400 kD).

A “reducing sugar” is a polyol which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Mannitol, xylitol, erythritol, threitol, sorbitol and glycerol are examples of sugar alcohols. As to sugar acids, these include L-gluconate and metallic salts thereof.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(.±.)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts that retain the biological effectiveness and properties of the compounds of this invention and, which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaricacid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

The term “therapeutically effective amount” or “effective amount” refers to that amount of a compound of Formula I that is sufficient to effect treatment, as defined below, when administered to a mammal in need of such treatment. More specifically, it's that amount that is sufficient to inhibit expressions of HIF-1 and HIF-1-regulated genes thereby inhibiting tumor angiogenesis, tumor growth and tumor progression and metastasis without side effects, e.g. apoptosis in cardiac myocyte. As used herein, “HIF-1-related genes” as used herein refer to the genes whose expressions are regulated by HIF-1. The following genes are included in this gene family; erythropoietin, transferrin, transferrin receptor, ceruloplasmin, vascular endothelial growth factor (VEGF), VEGF receptor FLT-1, transforming growth factor β3, plasminogen activator inhibitor 1, α1β adrenergic receptor, adrenomedullin, endothelin 1, nitric oxide synthase 2, heme oxygenase 1, glucose transporter 1 and 3, hexokinase 1 and 2, enolase 1, glyceraldehyde-3-phosphate dehydrogenase, phosphoglycerate kinase 1, phosphoglucokinase L, pyruvate kinase M, aldolase A and C, trios phosphate isomerase, lactate dehydrogenase A, carbonic anhydrase 9, adenylate kinase 3, propyl-4-hydroxylase a1, insulin-like growth factor (IGF) 2, IGF-binding protein 1, 2 & 3, P21, Nip3, cyclin G2 and differentiated embryo chondrocyte 1, The term “animal” as used herein is meant to include all mammals, and in particular humans. Such animals are also referred to herein as subjects or patients in need of treatment. The therapeutically effective amount will vary depending upon the subject and disease condition being treated, the weight and age of the subject, the severity of the disease condition, the particular compound of Formula I chosen, the dosing regimen to be followed, timing of administration, the manner of administration and the like, all of which can readily be determined by one of ordinary skill in the art.

The term “treatment” or “treating” means any treatment of a disease in a mammal, including:
a) preventing the disease, that is, causing the clinical symptoms of the disease not to develop;
b) inhibiting the disease, that is, slowing or arresting the development of clinical symptoms; and/or
c) relieving the disease, that is, causing the regression of clinical symptoms.

The active compounds according to the present invention may be administered by any suitable route, including orally, parenterally, by inhalation spray, rectally, or topically in dosage unit formulations containing conventional pharmaceutically acceptable carriers, adjuvants, and vehicles. The term “parenteral” as used herein includes, subcutaneous, intravenous, intraarterial, intramuscular, intrasternal, intratendious, intraspinal, intracranial, intrathoracic, infusion techniques or intraperitoneally. In preferred embodiments, the YC-1 is administered intraperitoneally. The dosage administered will be dependent upon the age, health, and weight of the recipient, kind of concurrent treatment, if any, frequency of treatment, and the nature of the effect desired.

Typcial pharmaceutically acceptable carriers include any non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; tale; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate. Coloring agents, releasing agents, coating agents, sweetening, flavoring and perfuming agents, preservatives and antioxidants can also be present in the pharmaceutical composition of the present invention according to the judgment of the formulator.

The composition comprising the active compound according to the present invention may be made up in dosage forms such as granules, powders, tablets, pills, capsules, solutions, suspensions, syrups, elixirs, emulsions, ointments, pastes, creams, lotions, gels, sprays, inhalants or patches. The composition of the present invention may be applied in a variety of solutions. Suitable solutions for use in accordance with the present invention are sterile, dissolve sufficient amounts of the active compound, and are not harmful for the proposed application. Methods of formulation are within the skill of pharmaceutical formulation chemists and are fully described in such works as Remington's Pharmaceutical Science, 18th Edition, Alfonso R. Gennaro, Ed., Mack Publishing Co., Easton, Pa., USA, 1990.

The YC-1,3-(5′-hydroxymethyl-2′-furyl)-1-benzylindazole, used in the present invention, may be manufactured by prior art techniques or is also available commercially. For example, YC-1 may be obtained from A.G. Scientific Inc. (San Diego, Calif.), Sigma RBI (St Louis, Mo., USA), or Alexis Biochemicals (San Diego, Calif.).

The compounds which are novel derivatives of YC-1 represented by Formula 1, inhibit the expressions of HIF-1α and the HIF-1-regulated genes in vitro and in vivo, are as follows: 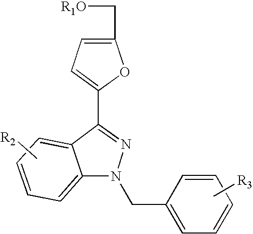
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
   including single isomers, mixtures of isomers, and pharmaceutically acceptable solvates and salts thereof.

The compounds of Formula 1 can be named and numbered (e.g., using AutoNom version 2.1) as described below. For example, the compound of Formula 1A: 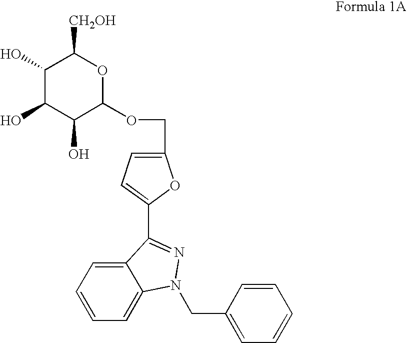
i.e., the compound according to Formula 1 where R¹ is mannose, i.e., 2-hydroxymethyl-tetrahydro-pyran-3,4,5,6-tetraol, R² is hydrogen and R³ is 4-hydrogen, can be named 2hydroxymethyl-6-0-[5-(1-Benzyl-indazol-3-yl)-furan-2-yl-methyl]-tetrahydro-pyran-3,4,5,6-tetraol.
Synthesis of the Compounds of Formula I

The compounds of the invention can be synthesized utilizing techniques well known in the art. Syntheses of the compounds of Formula 1 are illustrated below with reference to Reaction Scheme 1.

Synthetic Reaction Parameters

Unless specified to the contrary, the reactions described herein take place at atmospheric pressure, generally within a temperature range from −10° C. to 110° C. Further, except as employed in the Examples or as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

The terms “solvent”, “organic solvent” or “inert solvent” each mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can, of course, also be used.

When desired, the (R)- and (S)-isomers may be resolved by methods known to those skilled in the art, for example by formation of diastereoisomeric salts or complexes which may be separated, for example, by crystallisation; via formation of diastereoisomeric derivatives which may be separated, for example, by crystallisation, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. For example, a compound of Formula I can be dissolved in a lower alkanol and placed on a Chiralpak AD (205×20 mm) column (Chiral Technologies, Inc.) conditioned for 60 min at 70% EtOAc in Hexane. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting one enantiomer to the other by asymmetric transformation.

Starting Materials

The R¹-alcohols of Formula 101 and the like are commercially available, e.g., from Aldrich Chemical Company, Milwaukee, Wis. Other reactants are likewise commercially available or may be readily prepared by those skilled in the art using commonly employed synthetic methodology. 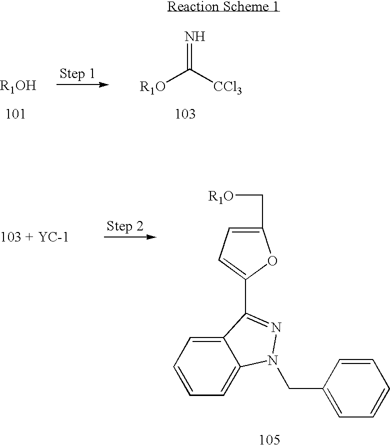
Preparation of Compounds of Formula 103

Referring to Reaction Scheme 1, Step 1, an alcohol of the Formula R₁OH is treated with trichloroacetonitrile and a base such as potassium carbonate in a nonpolar, aprotic solvent such as methylene chloride. The solution is stirred for 1-96 hours at room temperature to 150° C. to afford the corresponding compound of Formula 103, which is conventionally isolated and purified.

Preparation of Compounds of Formula 105

Referring to Reaction Scheme 1, Step 2, a compound of Formula 103 is treated with trimethylsilylmethyl trifluoromethanesulfonate (TMSOTf) and YC-1 in a nonpolar, aprotic solvent such as methylene chloride at about −30° C. The solution is stirred for 1-96 hours at between −30° C. and room temperature to afford the corresponding compound of Formula 105, which is conventionally isolated and purified.

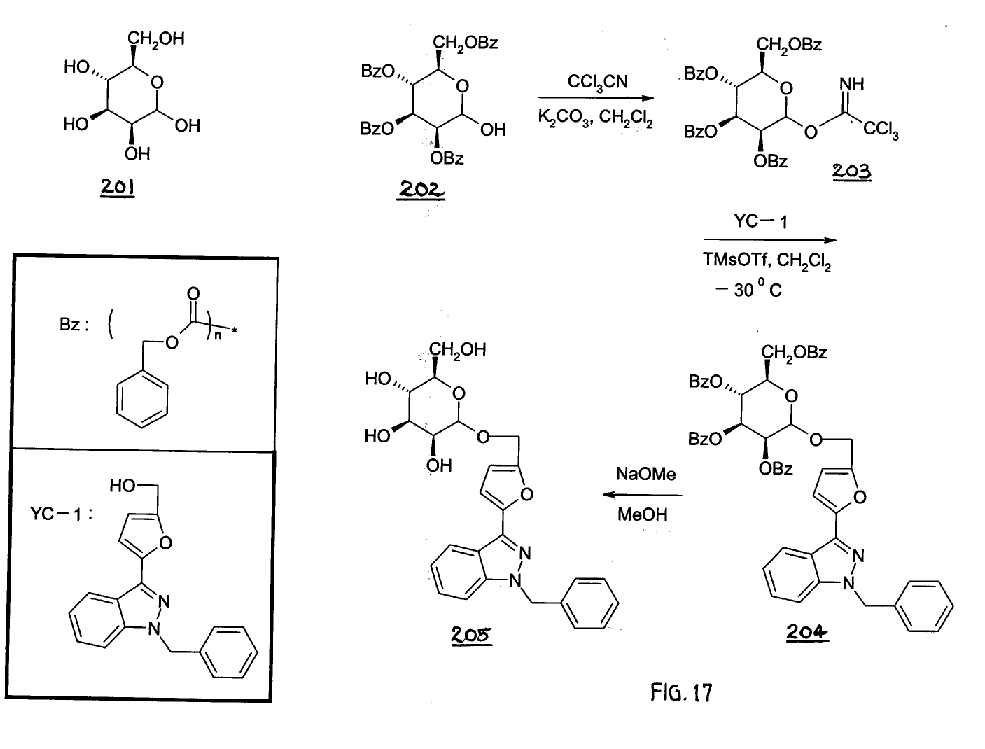

Optionally, any protecting groups are then removed. For example, referring to FIG. 17, in a particular embodiment, R₁₀H is 2-hydroxymethyl-tetrahydro-pyran-3,4,5,6-tetraol (201) wherein one (and preferably all of the hydroxyls other than the anomeric hydroxyl) are protected with a suitable protecting group, preferably benzyloxycarbonyl, as in compound 202. The activated sugar 203 is then reacted with YC-1 to produce compound 204. In such an instance, the protecting group may be removed by treatment with base, preferably with sodium methoxide in methanol to produce the YC-1 sugar derivative 205.

Compounds prepared by the above-described process of the invention can be identified, e.g., by the presence of a detectable amount of Formula 103 or YC-1. While it is well known that pharmaceuticals must meet pharmacopoeia standards before approval and/or marketing, and that synthetic reagents and precursors should not exceed the limits prescribed by pharmacopoeia standards, final compounds prepared by a process of the present invention may have minor, but detectable, amounts of such materials present, for example at levels in the range of 95% purity with no single impurity greater than 1%. These levels can be detected, e.g., by emission spectroscopy. It is important to monitor the purity of pharmaceutical compounds for the presence of such materials, which presence is additionally disclosed as a method of detecting use of a synthetic process of the invention.

Preferred Processes and Last Steps

A racemic mixture of isomers of a compound of Formula 1 is placed on a chromatography column and separated into (R)- and (S)-enantiomers.

A compound of Formula 1 is contacted with a pharmaceutically acceptable base to form the corresponding base addition salt.

A pharmaceutically acceptable acid addition salt of Formula 1 is contacted with an acid to form the corresponding compound of Formula 1. A compound of Formula 1 is contacted with a pharmaceutically acceptable acid to form the corresponding acid addition salt.

A pharmaceutically acceptable acid addition salt of Formula 1 is contacted with a base to form the corresponding free base of Formula 1.

Preferred Compounds

Preferred for the compounds, pharmaceutical formulations, methods of manufacture and use of the present invention are the following combinations and permutations of substituent groups of Formula 1 (sub-grouped, respectively, in increasing order of preference).

In one embodiment, R₁ is derived from a reducing sugar. More particularly, R₁ is derived from fructose, mannose, maltose, lactose, arabinose, xylose, ribose, rhamnose, galactose and glucose. Yet more particularly, R₁ is derived from mannose.

In one embodiment, R₂ and R₃ are independently hydrogen, hydroxy, cyano, halogen (preferably fluoro or chloro), optionally substituted lower-alkoxy (preferably methoxy), optionally substituted lower-alkyl (preferably methyl or trifluoromethyl), carbamoyl, carboxy, or acyl.

Utility, Testing and Administration

Utility

The present invention is based on the surprising discovery that compounds of Formula 1 exhibit an anticancer effect in vivo by blocking tumor angiogenesis essential for tumor growth and metastasis.

Accordingly, one aspect of the present invention provides a method of inhibiting HIF-1α expression in tumor cells or tissues, comprising contacting the tumor cells or tissues with a composition comprising a compound of Formula 1 at an effective amount for inhibiting HIF-1α.

Another aspect of the present invention provides a method of inhibiting HIF-1-regulated gene expression in tumor cells or tissues, comprising contacting the tumor cells or tissues with a composition comprising a compound of Formula 1 at an effective amount for inhibiting HIF-1-regulated gene expression.

The other aspect of the present invention provides a method of inhibiting angiogenesis in tumor cells or tissues, comprising contacting the tumor cells or tissues with a composition comprising a compound of Formula 1 at an effective amount for inhibiting angiogenesis.

A further aspect of the present invention provides a method of inhibiting tumor growth in animal tissues, comprising contacting the animal tissues with a composition comprising a compound of Formula 1 at an effective amount for inhibiting tumor growth.

Yet another aspect of the present invention provides a method of inhibiting tumor progression and metastasis in animal tissues, comprising contacting the animal tissues with a composition comprising a compound of Formula 1 at an effective amount for inhibiting tumor progression and metastasis.

A further aspect of the present invention provides a method of treating a HIF-1-mediated disorder or condition in a mammal, comprising administering to the mammal a composition including a therapeutically effective amount of a compound of Formula 1.

The present invention is broadly applicable to a variety of uses which include inhibition of angiogenesis induced by HIF-1 and treatment of HIF-1-mediated disorders or conditions with accompanying undesired angiogenesis, such as solid and blood-borne tumors including but not limited to melanomas, carcinomas, sarcomas, rhabdomyosarcoma, retinoblastoma., Ewing sarcoma, neuroblastoma, osteosarcoma, and leukemia.

Testing

YC-1 has been shown to have an inhibitory effect on the expression of HIF-1α and on the induction of VEGF, aldolase A, and enolase 1 in cancer cells cultured under hypoxic conditions. In vivo, treatment with YC-1 halted the growth of xenografted tumors originating from hepatoma, stomach carcinoma, renal carcinoma, cervical carcinoma, and neuroblastoma cells. Tumors from YC-1-treated mice showed fewer blood vessels and reduced expression of HIF-1α protein and HIF-1-regulated genes than tumors from vehicle-treated mice. These results support that YC-1 is an inhibitor of HIF-1 that halts tumor growth by blocking tumor angiogenesis and tumor adaptation to hypoxia.

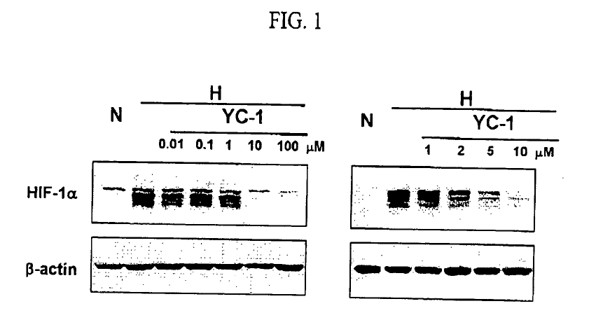

Compounds of Formula 1 can be evaluated for efficacy using the methods described above with regard to YC-1. In addition, a compound of Formula 1 has been shown to have efficacy in a cellular proliferation assay using Hep3B (liver cancer cells). The cells were treated with a compound of Formula 1 (at concentrations ranging from 30-60 mg/kg) and buffer. Cellular proliferation was measured at 72 hours. The results are shown in FIG. 1. Notably treatment with the compound resulted in a notable decrease in tumor size.

Administration

The compounds of Formula 1 are administered at a therapeutically effective dosage, e.g., a dosage sufficient to provide treatment for the disease states previously described. While human dosage levels have yet to be optimized for the compounds of the invention, generally, a daily dose is from about 0.05 to 100 mg/kg of body weight, preferably about 0.10 to 10.0 mg/kg of body weight, and most preferably about 0.15 to 1.0 mg/kg of body weight. Thus, for administration to a 70 kg person, the dosage range would be about 3.5 to 7000 mg per day, preferably about 7.0 to 700.0 mg per day, and most preferably about 10.0 to 100.0 mg per day. The amount of active compound administered will, of course, be dependent on the subject and disease state being treated, the severity of the affliction, the manner and schedule of administration and the judgment of the prescribing physician; for example, a likely dose range for oral administration would be about 70 to 700 mg per day, whereas for intravenous administration a likely dose range would be about 700 to 7000 mg per day, the active agents being selected for longer or shorter plasma half-lives, respectively.

Administration of the compounds of the invention or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administration are customary in treating the indications that are the subject of the present invention.

Pharmaceutically acceptable compositions include solid, semi-solid, liquid and aerosol dosage forms, such as, e.g., tablets, capsules, powders, liquids, suspensions, suppositories, aerosols or the like. The compounds can also be administered in sustained or controlled release dosage forms, including depot injections, osmotic pumps, pills, transdermal (including electrotransport) patches, and the like, for prolonged and/or timed, pulsed administration at a predetermined rate. Preferably, the compositions are provided in unit dosage forms suitable for single administration of a precise dose.

The compounds can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical formulation will contain about 0.005% to 95%, preferably about 0.5% to 50% by weight of a compound of the invention. Actual methods of preparing such dosage forms are known, or will be apparent, to those skilled in this art; for example, see Remington's Pharmaceutical Sciences, Mack Publishing Company, Easton, Pa.

In addition, the compounds of the invention can be co-administered with, and the pharmaceutical compositions can include, other medicinal agents, pharmaceutical agents, adjuvants, and the like. Suitable additional active agents include, for example: with alkylating agents such as asaley, AZQ, BCNU, busulfan, carboxyphthalatoplatinum, CBDCA, CCNU, CHIP, chlorambucil, chlorozotocin, cis-platinum, clomesone, cyanomorpholinodoxorubicin, cyclodisone, dianhydrogalactitol, fluorodopan, hepsulfam, hycanthone, melphalan, methyl CCNU, mitomycin C, mitozolamide, nitrogen mustard, PCNU, piperazine alkylator, piperazinedione, pipobroman, porfiromycin, spirohydantoin mustard, teroxirone, tetraplatin, thio-tepa, triethylenemelamine, uracil nitrogen mustard and Yoshi-864; antimitotic agents such as allocolchicine, Halichondrin B, colchicines, colchicine derivatives, dolastatin 10, maytansine, rhizoxin, taxol, taxol derivatives, thiocolchicine, trityl cysteine, vinblastine sulfate and vincristine sulfate; topoisomerase I inhibitors such as camptothecin, camptothecin derivatives, aminocamptothecin and morpholinodoxorubicin; topoisomerase II inhibitors such as doxorubicin, amonafide, m-AMSA, anthrapyrazole derivatives, pyrazoloacridine, bisantrene, daunorubicin, deoxydoxorubicin, mitoxantrone, menogaril, N,N-dibenzyl daunomycin, oxanthrazole, rubidazone, VM-26 and VP-16; and antimetabolites such as L-alanosine, 5-azacytidine, 5-fluorouracil, acivicin, aminopterin derivatives, antifol, Baker's soluble antifol, dichlorallyl lawsone, brequinar, ftorafur, 5,6-dihydro-5-azacytidine, methotrexate, methotrexate derivatives, N-(phosphonoacetyl)-L-aspartate (PALA), pyrazofurin, trimetrexate, 3-HP, 5-HP, 2′-deoxy-5-fluorouridine, alpha-TGDR, aphidicolin glycinate, ara-C, 5-aza-2′deoxycytidine, beta-TGDR, cyclocytidine, guanazole, hydroxyurea, inosine glycodialdehyde, macbecin II, pyrazoloimidazole, thioguanine and thiopurine.

In one preferred embodiment, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. an active compound as defined above and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of active compound contained in such parenteral compositions is highly dependent on the specific nature thereof, as well as the activity of the compound and the needs of the subject. However, percentages of active ingredient of 0.01% to 10% in solution are employable, and will be higher if the composition is a solid which will be subsequently diluted to the above percentages. Preferably the composition will comprise 0.2-2% of the active agent in solution.

Formulations of the active compound or a salt may also be administered to the respiratory tract as an aerosol or solution for a nebulizer, or as a microfine powder for insufflation, alone or in combination with an inert carrier such as lactose. In such a case, the particles of the formulation have diameters of less than 50 microns, preferably less than 10 microns.

The present invention is more specifically illustrated by the following examples. However, it should be understood that these examples are provided only for illustration of the present invention, but not intended to limit the present invention in any manner,

### EXAMPLES

### Example 1

Materials

YC-1,3-(5′-hydroxymethyl-2′-furyt)-1-benzylindazole, was purchased from A.G. Scientific Inc. (San Diego, Calif.), resuspended in DMSO at a stock concentration of 120 mg/ml, and stored at −30*C. All culture media and fetal bovine serum (FBS) were purchased from Life Technologies (Grand Island, N.Y.).

### Example 2

Cell Culture

The Hep3B hepatoma, Caki-I renal carcinoma, SiHa cervical carcinoma, and SK-N-MC neuroblastoma cell lines were obtained from the American Type Culture Collection (Manassas, Va.). The NCI-H87 stomach carcinoma cell line was obtained from the Korean Cell Line Bank (Seoul, Korea). Hep3B cells were cultured in a modified Eagle's medium, Caki-1, SiHa., and SK-N-MC cells in Dulbecco's modified Eagle's medium, and NCI-H87 cells in RPM1 1640 medium. All culture media were supplemented with 10% heat-inactivated FBS, 100 units/mL penicillin, and 100 Vg/mL streptomycin. All cells were grown in a humidified atmosphere containing 5% CO₂ at 37° C., in which the oxygen tension in the incubator (Vision Sci Co., model 9108MS2, Seoul, KOREA) was held at either 140 mm Hg (20%02, v/v, normoxic conditions) or 7 mm Hg G % O₂, v/v, hypoxic conditions). YAC-1 cell line was obtained from the American Type Culture Collection and maintained in RPMI 1640 medium supplemented with 10% FBS, 1% L-glutamine, 1% nonessential amino acids, 1% sodium pyruvate, 100 units/mL penicillin, and 100˜Lg/ml, streptomycin.

### Example 3

Effect of YC-1 on the Expressions of HIF-α and HIF-1-Regulated Genes in Hep3B Hepatoma Cells

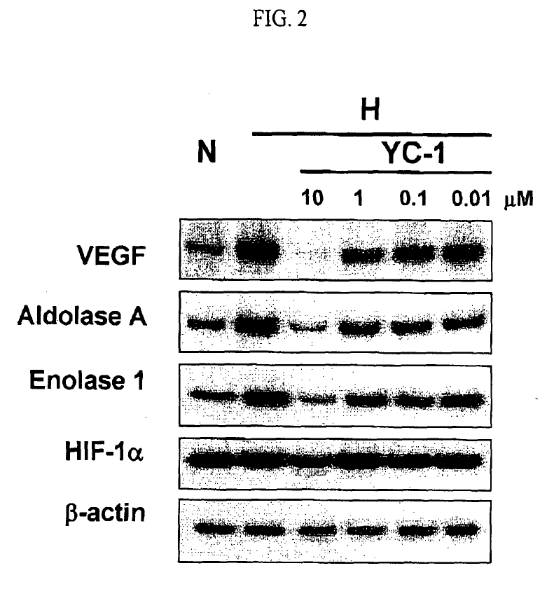

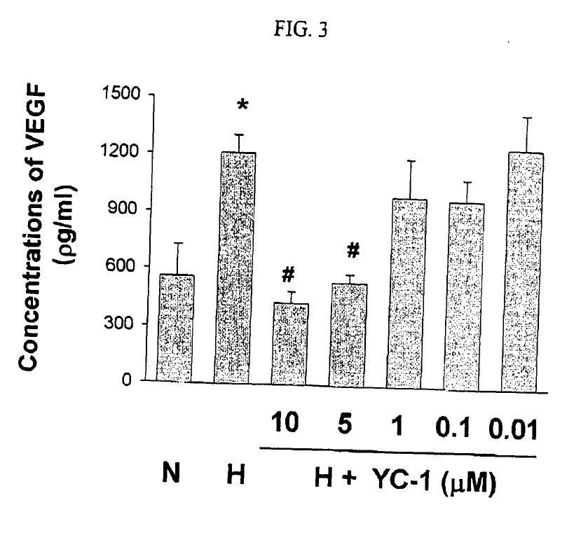

To investigate the inhibitory effect of YC-1 on HIF-1-mediated hypoxic responses, Hep3B cells were treated with YC-1 under hypoxic conditions. Hep3B cells were treated with the indicated concentrations of YC-1 for 5 minutes before being cultured for 4 hours under normoxic (N, 20%02 V/V) or hypoxic (H, 1% O₂ V/V) conditions. Immunoblotting was used to detect HIF-1 protein in cultured cells, as described (Chun et al., Biochem Pharmacol 2001 61:947-954). Cells were centrifuged at 3000 rpm for 5 minutes at VC and then washed twice with ice-cold phosphate buffered saline (PBS). Cells were then resuspended in ten packed cell volumes of alysis buffer consisting of 10 mM Tris, pH 7.4, 130 mM NaCl, 2 mM EDTA, 1% Nonidet P-40, 0.5 mM dithiothreitol, and 0.5 mM phenylmethylsulfonyl fluoride. Proteins (20 μg) in the cell extract was separated on 6.5% SDS/polyacrylamide gels, and then transferred to an Immobilon-P membrane (Millipore, Bedford, Mass.). Membranes were blocked with 5% nonfat milk in tris-buffered saline containing 0.1% Tween-20 (TTBS) at room temperature for 1 hour and then incubated overnight at 4*C with rabbit anti-HIF-1 a (Chun et al., J Cell Sci 2001 114:4051-406 1) diluted 1:1000 in 5% nonfat milk in TTBS. Horseradish peroxidase-conjugated anti-rabbit antiserum (Zymed Laboratories Inc., South San Francisco, Calif.) was used as a secondary antibody (1:5000 dilution in 5% nonfat milk in TTBS, 2 hours incubation) and the antigen antibody complexes were visualized by using an Enhanced Chemiluminescence Plus kit (Amersham Biosciences Corp., Piscataway, N.J.). As a result, the HIF-1α protein level increased in cells cultured under these conditions for 4 hours without YC-1 but dose dependently decreased in cells cultured with YC-1 (FIG. 1). To quantify mRNAs for HIF-1α and HIF-1-regulated genes, we performed a highly sensitive, semi-quantitative reverse transcription-polymerase chain reaction (RT PCR), as described previously (Chun et al., J Cell Sci 2001 114:4051-4061). Hep3B cells were treated with the indicated concentrations of YC-I for 5 minutes before being cultured for 16 hours under normoxic (N, 20% O₂ V/V) or hypoxic (H, 1% O₂ V/V) conditions. Total RNAs were isolated from cultured cells using TRIZOL (Life Technologies). After verifying the RNA quality on a 1% denaturing agarose gel, one jig of total RNA was added to a 50-pL RT-PCR reaction mixture, containing 5 μCi [a ³²P]dCTP (NEN Life Science, Boston, Mass.) and 250 nM of each primer pair. The RT PCR was performed using one cycle of reverse transcription at 48° C. for 1 hour and then 18 PCR cycles, in which one cycle consisted of a denaturation step at 94° C. for 30 seconds, an annealing step at 53° C. for 30 seconds, and an elongation step at 68° C. for 1 minute. The resulting PCR fragments (5 gL) were electrophoresed through a 4% polyacrylamide gel at 120 V in a 0.3× Tris-Borate-EDTA (TBE) buffer at VC. The gels were dried and then autoradiographed. P-actin mRNA was measured as a PCR control. The nucleotide sequences of the primer pairs (5′ to 3′) were AACTTTCTGCTGTCTTGG (SEQ ID NO: 1) and TTTGGTCTGCATTCACAT (SEQ ID NO: 2) for VEGF, GTCATCCTCTTCCATGAGAC (SEQ ID NO: 3) and AGGTAGATGTGGTGGTCACT (SEQ ID NO: 4) for aldolase A, AAGAAACTGAACGTCACAGA (SEQ ID NO: 5) and GATCTTCGATAGACACCACT (SEQ ID NO: 6) for enolase 1, CCCCAGATTCAGGATCAGACA (SEQ ID NO: 7) and CCATCATGTTCCATTTTTCGC (SEQ ID NO: 8) for HIF-1α, and AAGAGAGGCATCCTCACCCT (SEQ ID NO: 9) and ATCTCTTGCTCGAAGTCCAG (SEQ ID NO: 10) for β-actin. As a result, the expression of HIF-1-regulated genes (VEGF, aldolase A, and enolase 1) dose dependently decreased in cells cultured with YC-1 for 16 hours, whereas the expression of β-actin mRNA was not affected (FIG. 2). The HIF-1a mRNA level was also relatively unchanged in cells cultured with YC-1, suggesting that YC-1-mediated inhibition of HIF-I a occurs at a post-transcriptional level. To assess whether the VEGF mRNA levels affected levels of VEGF protein secreted into the medium, we measured VEGF protein levels in Hep3B cell-conditioned medium. For this, Hep3B were plated in a 6-well plate at a density of 1×10′ cells/well in a-modified Eagle's medium supplemented with 10% heat-inactivated FBS and incubated overnight. Cells were treated with YC-1 or vehicle (DMSO) for 5 minutes before cells were subjected to normoxia or hypoxia for 24 hours. VEGF levels in the conditioned media were quantified by using the Quantikine human VEGF Immunoassay kit (R&D Systems, Minneapolis, Minn.) according to the manufacturer's recommended protocol. The VEGF concentrations were quantified by comparison with a series of VEGF standard samples included in the assay kit. After 24 hours, the VEGF protein level in medium from cells cultured under hypoxic conditions (mean=1208 pg/ml, 95% CI=1112 to 13 04 pg/ml, P<0.00 1 versus normoxic conditions) was more than twice that from cells cultured under normoxic conditions (mean=559 pg/ml, 95% CI=392 to 26 pg/ml) (FIG. 3). Compared with the VEGF protein level in medium from untreated cells grown under hypoxic conditions, the VEGF protein level in medium from cells cultured with YC-I decreased in a dose-dependent manner (P<0.001 versus untreated hypoxic conditions) (FIG. 3).

### Example 4

Effect of YC-1 on the Expression of HIF-1α and VEGF in Cancer Cells of Different o

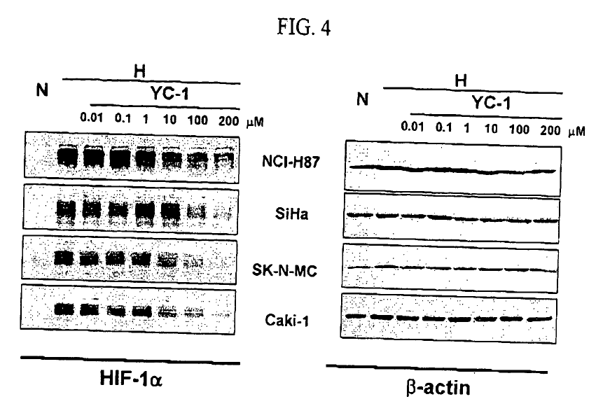

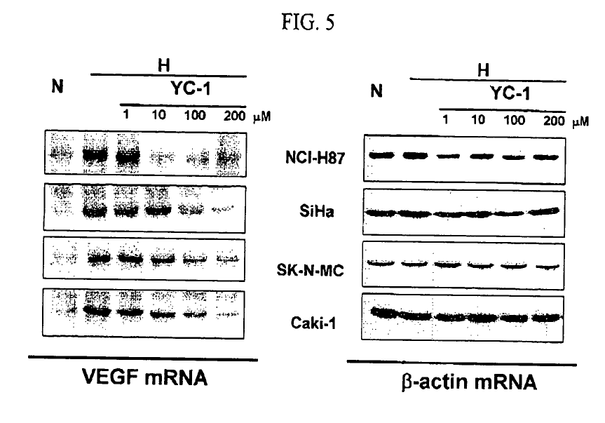

We examined whether the effects of YC-1 were specific to Hep3B cells by assessing the expression of HIF-1α protein and VEGF mRNA in other tumor cell lines (NCI-H87, SiHa, SK-N-MC, and Caki-1) cultured under hypoxic conditions in the absence or presence of YC-I. NCI-H87 gastric carcinoma, SiHa cervical carcinoma, SK-N-MC neuroblastoma, and Caki-I renal carcinoma cells were treated with the indicated concentrations of YC-I for 5 minutes before being cultured under normoxic (N, 20%02 V/V) or hypoxic (H, 1% O₂ V/V) conditions for 4 hours. Levels of HIF-1α and β-actin proteins were analyzed by immunoblot analysis using a rabbit anti-HIF-1α antibody or a rabbit anti-p-actin antibody. Proteins were visualized by enhanced chemiluminescence. HIF-1 protein and VEGF mRNA were induced in all cell lines cultured under hypoxic conditions in the absence of YC-1 (FIGS. 4 and 5). The levels of HIF-1α protein and VEGF mRNA were dose-dependently inhibited in cells cultured under hypoxic conditions in the presence of YC-1. These results confirm that YC-1 inhibits the HIF-1-mediated induction of hypoxia-inducible genes, regardless of the tumor cell type.

### Example 5

Effects of YC-1 on Tumor Growth In Vivo

Because of the observed in vitro effects of YC-1, we investigated whether YC-1 inhibits angiogenesis in solid tumors by suppressing HIF-1, and thus inhibits tumor growth in vivo.

Male nude (BALB/cAnNC˜-nu/nu) mice were purchased from Charles River Japan Inc. (Shin-Yokohama, JAPAN). The animals were housed in a specific pathogen-free room under controlled temperature and humidity. All animal procedures were performed according to the established procedures of the Seoul National University Laboratory Animal Maintenance Manual. Eighty mice aged 7-8 weeks were injected with tumor cells for the xenograft experiments. Sixty nine mice bearing tumors were used for the experiments, but eleven mice were excluded because of technical problems associated with the injection or lack of tumor growth. Twenty five mice were injected subcutaneously in the flank with 5×10⁶ viable Hep3B cells. The mice were immediately randomly assigned to one of three groups. The first group (n=12) was a control group and received the vehicle (DMSO). The second group (n 7) received daily intraperitoneal injections of YC-1 (30 μg) beginning the day after the injection of Hep3B cells and continuing for 2 weeks. The third group (n=6) received daily intraperitoneal injections of YC-1 (30 gg/g) for 2 weeks after the Hep3B tumors measured 100-150 mm³. In other experiments, forty four mice were injected with 5×10⁶ NCI-1187, SiHa, SK-N-MC, and Caki-1 tumor cells. Of the mice in each group, 13, 10, 10, and 11, respectively, developed tumors. The tumor-bearing mice were randomly assigned to either a control group or an experimental group. After the tumors reached an approximate volume of 100-150 mm³, the mice in the experimental group received daily intraperitoneal injections of YC-1 (30 pg/g) for 2 weeks.

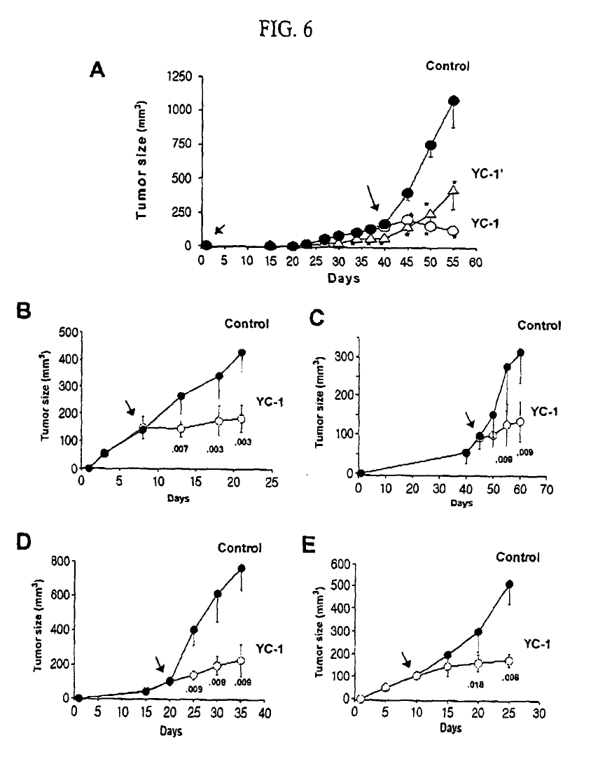

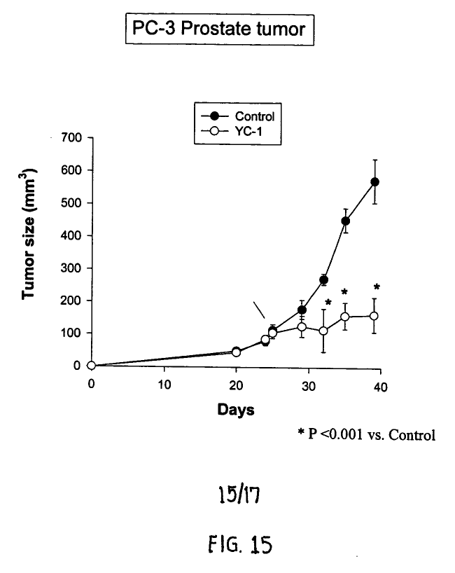

Tumors were measured every 2 or 3 days with calipers in two dimensions and the tumor volumes were calculated using the formula: Volume=axb2/2, where a is the width at the widest point of the tumor and h is the width perpendicular to a. The results from individual mice were plotted as average tumor volume versus time. As a consequence, tumors in YC-1-treated mice were visibly smaller than those in vehicletreated mice (FIG. 6). Hep3B tumor growth was minimal in mice treated with YC-1 the day after the tumor cells were injected (the last day of experiment, mean=422 mM³′95% CI 283 to 561 mrn˜, P<0.001 versus vehicle-treated group [mean=1082 mm 95% CI 880 to 1284 and was halted in mice treated with YC-1 after the tumors had become established (mean=126 mm³, 95% Cl=97 to 155 mm, P<0.001 versus vehicle-treated group) (FIG. 6, A). Tumors in mice bearing NCI-H87 (FIG. 6, B), SiHa (FIG. 6, C), SK-N-MC (FIG. 6, D), and Caki-I (FIG. 6, E) xenografts were also statistically significantly smaller in mice treated with YC-1 than in mice treated with the vehicle (P<0.01 for all comparisons). Similar tests were performed using instead viable PC-3 human prostate tumor cells. The results are showin in FIG. 15. These results indicate that YC-1 effectively inhibits tumor growth and tumor progression in tumor-bearing mice.

### Example 6

### Effects of YC-1 on Angiogenesis, HIF-1α Protein, and VEGF Expression

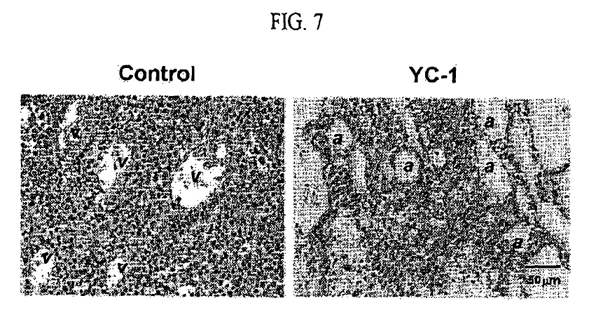

To determine the mechanism by which YC-1 inhibits tumor growth, we examined Hep3B tumors morphologically and biochemically. Male nude mice were injected subcutaneously in the flank with 5×10⁶ viable Hep3B cells. After the tumors reached 100 to 150 mm³ in size, mice received an intraperitoneal injection of YC-1 (30 μ/g) or vehicle (DMSO) daily for 2 weeks. After the last treatment, the mice were euthanized, the tumors, removed, fixed with formalin, and embedded in paraffin. Serial sections (6-˜Lm thick) were cut from each paraffin block. One section was stained with hematoxylin and eosin (H&E) for histological assessment. Hematoxylin-eosin stained tumor sections from vehicle-treated mice revealed well-developed blood vessels containing red blood cells and frequent mitotic figures (FIG. 7). By contrast, hematoxylin-eosin stained tumor sections from YC-1-treated mice tumors revealed frequent acinus formation without well-developed blood vessels (FIG. 7).

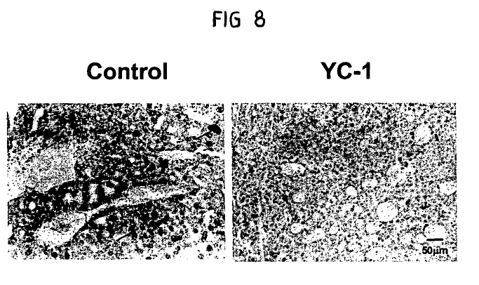

To determine whether the inhibitory effect of YC-1 on tumor growth is associated with the suppression of tumor angiogenesis, we examined the distribution of the endothelial marker, CD31. Other sections were immunochemically stained for HIF-1 and the endothelial cell marker CD31. First, the sections were deparaffinized and rehydrated through a graded alcohol series. Next, the sections were heated in 10 mM sodium citrate (pH 6.0) for 5 minutes in a microwave to retrieve the antigens. After blocking nonspecific sites with a blocking solution containing 2.5% BSA (SigmaAldrich Corp., St. Louis, Mo.) and 2% normal goat serum (Life Technologies) in a phosphate-buffered saline (pH 7.4) for 1 hour, the sections were incubated overnight at 4° C. with rabbit polyclonal anti-CD31 (SantaCruz, 1:100 dilution in the blocking solution) or rat anti-HIF-1α (1:100 dilution in the blocking solution) antibodies, as described previously (Kim et al., Circ Res 2002 90:E25-E33). Negative control sections were incubated with diluent in the absence of any primary antibodies. The sections were then stained using standard methods, and the avidin-biotin-horseradish peroxidase complex was used to localize the bound antibodies, with diaminobenzidine as the final chromogen. All immunostained sections were lightly counterstained with hematoxylin. Few CD31-immunopositive vessels were observed in tumor sections from YC-1-treated mice, whereas many vessels were observed in tumor sections from vehicle-treated mice (FIG. 8).

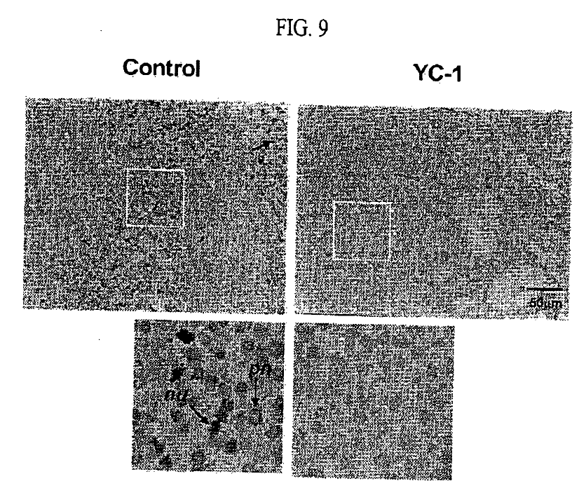

Because HIF-1 is important in angiogenesis, we next assessed HIF-1α expression in tumor sections from vehicle- and YC-1-treated mice (FIG. 9). Hep3B tumors from vehicle-treated mice showed nuclear immunoreactivity (nu) and perinuclear immunoreactivity (pn) for HIF-1α, but only in relatively hypoxic areas away from blood vessels. However, tumor sections from YC-1-treated mice showed no HIF-1α inimunoreactive cells (FIG. 9).

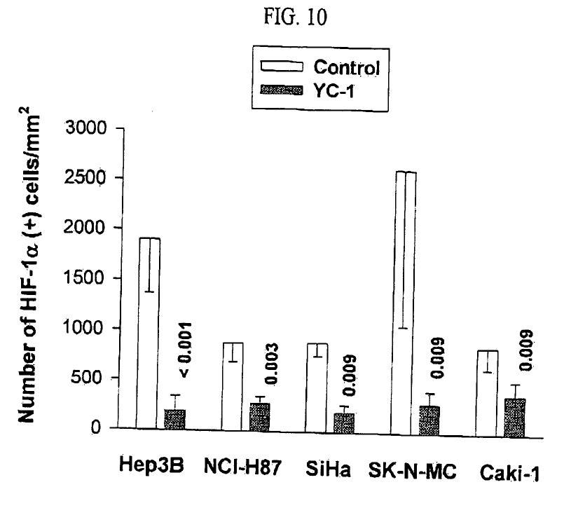

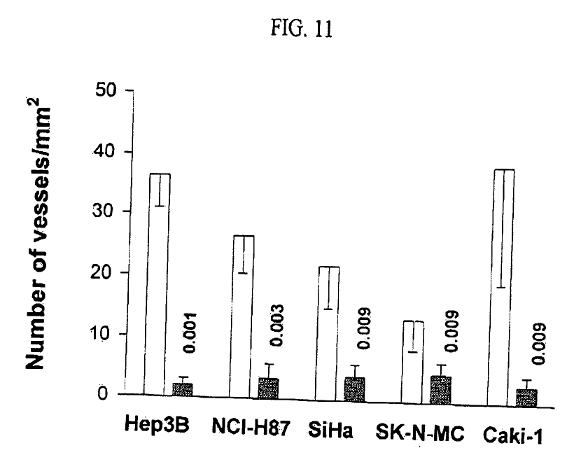

We further quantified the numbers of HIF-1α-positive cells and CD31-positive vessels in tumor sections from vehicle- and YC-1-treated mice. For histological assessment, HIF-1α-positive cells and CD31-positive vessels were identified at magnifications of 200× and 100×, respectively, and examined using a Sony XC-77 CCD camera and a Microcomputer Imaging Device model 4 (MCID-M4) image analysis system. The expression of HIF-1α and the vessel density were measured by counting the numbers of immunopositive cells and vessel profiles (identified by CD31 staining) per mm² under microscopic images. We analyzed ten or more different lesions per xenograft tumor. Regardless of cell origin, the expression of HIF-1α protein and blood vessel formation were statistically significantly inhibited in mice treated with YCI for 2 weeks (P<0.01 for all comparisons) (FIGS. 10 and 11).

### Example 7

Effect of YC-1 on the eARression of HIF-1α and HIF-1-Regulated Genes in Hep3B Hepatoma Cell Xenografts

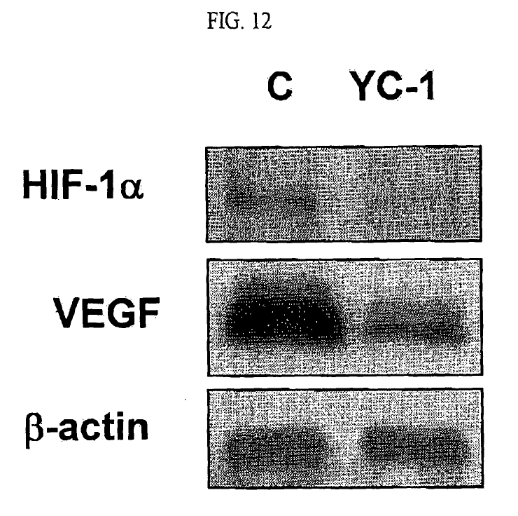

To confirm the effects of YC-1 on the expression HIF-1α and VEGF, we isolated the protein from Hep3B tumor by immunoprecipitation and immunoblotting. Male nude mice were injected subcutaneously in the flank with 5×10⁶ viable Hep3B cells. After the tumors reached 100 to 150 mm³ in size, mice received an intraperitoneal injection of YC-1 (30˜μg/g) or vehicle (DMSO) daily for 2 weeks. After the last treatment, the mice were euthanized, the tumors removed and lysates prepared for immunoblotting. Tumor lysates from vehicle-treated mice (C) and from. YC-1-treated (YC-1) mice were assessed by immunoblotting for HIF-1α and VEGF protein levels. For the immunoprecipitation of HIF-1α in tumor tissues, tissue lysates in the lysis buffer (150 μg protein) were incubated with 10-μL of the rabbit anti-HIF-1α antiserurm overnight at 4° C., and then incubated with protein A-Sepharose beads (Amersham Biosciences Corp.) at a dilution of 1:100 for 2 hours. The antigen-antibody-protein A complexes were washed extensively with the lysis buffer, the immunocomplexes were eluted by boiling for 3 minutes in a sample buffer containing 2% SDS and 10 mM dithiothreitol and subjected to SDS-PAGE, and then immunoblotted using a rat anti HIF-1α antibody developed previously (Chun et al., Biochem J 2002 362:71-79). β-actin protein was measured as an internal standard. VEGF in tumor tissue was detected using a mouse monoclonal anti-VEGF (SantaCruz Biotechnology Inc., Santa Cruz, Calif.) at a dilution of 1:1000, followed by incubation with a horseradish peroxidase-conjugated anti-mouse antiseruni (Zymed Laboratories Inc.). The levels of HIF-1α and VEGF protein expressions were markedly lower in YC-1-treated tumors than in vehicle-treated tumors (FIG. 12).

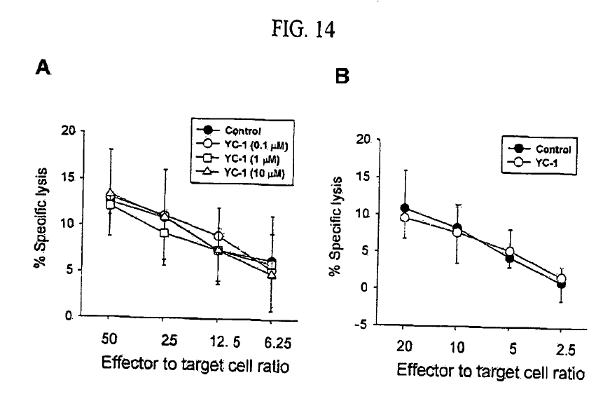

To confirm the effects of YC-1 on the expression of HIF-1-regulated genes in Hep3B hepatoma, cell xenografts, we measured the mRNA levels of VEGF, aldolase A, and enolase I by semi-quantitative RT-PCR according to the same method as described in EXAMPLE 3. The quality of the extracted RNAs was verified by identifying the 18S ribosomal RNA (rRNA) on a 1% denaturing agarose gel. VEGF, aldolase A and enolase 1 mRNA levels were also lower in YC-1-treated tumors than in vehicle-treated tumors one experiment. Results are expressed as the mean and 95% confidence intervals of 4 separate experiments. Splenic lymphocytes incubated with YC-1 in vitro had cytolytic activity against NK-cell sensitive YAC-1 cells that was comparable to that from splenic lymphocytes incubated without YC-1 (FIG. 14, A).

To examine the in vivo effect of YC-1 on NK cell activity, mice received a daily intraperitoneal injection of DMSO (n=4) or of YC-1 (30 μg/g, n=4) for 2 weeks. Splenic lymphocytes were isolated from each mouse and tested immediately for NK cell activity. The spontaneous release of ⁵¹Cr from YAC-1 cells was usually lower than 10% of the total ⁵¹Cr loaded. NK cell activity was calculated as follows: {(experimental release−spontaneous release)/(total release−spontaneous release)}×100. Each assay was repeated three times and the average value is the result from one experiment. Results are expressed as the mean and 95% confidence intervals of 4 separate experiments. Splenic lymphocytes from mice treated with YC-1 for 2 weeks had cytolytic activity that was comparable to that from vehicle-treated mice (FIG. 14, B).

### Example 9

Cell Culture

The Hep3B hepatoma was obtained from the American Type Culture Collection (Manassas, Va.). Hep3B cells were cultured in α-modified Eagle's medium. All culture media were supplemented with 10% heat-inactivated FBS, 100 units/mL penicillin, and 100 μg/mL streptomycin. All cells were grown in a humidified atmosphere containing 5% CO₂ at 37° C., in which the oxygen tension in the incubator (Vision Sci Co., model 9108MS2, Seoul, KOREA) was held at either 140 mm Hg (20% O₂, v/v, normoxic conditions) or 7 mm Hg (1% O₂, V/V, hypoxic conditions).

### Example 10

Effect of Compounds of Formula 1 on Hep3B Hepatoma Cell Xenografts

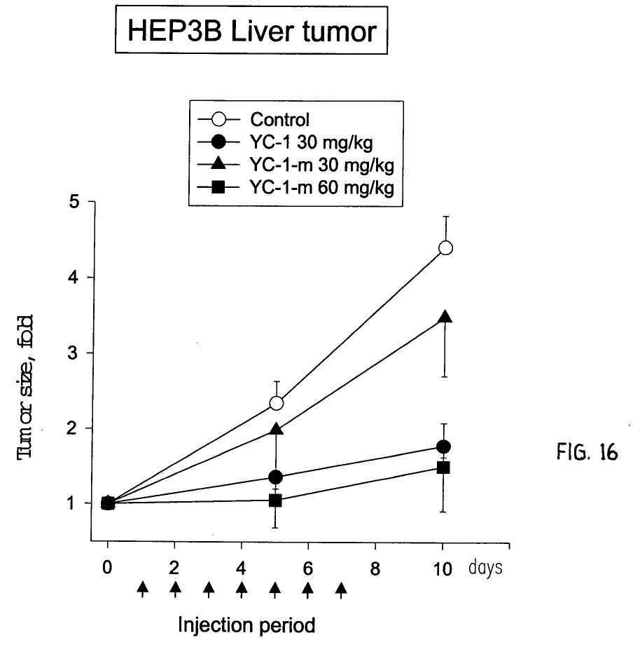

Male nude mice were injected subcutaneously in the flank with 5×10⁶ viable Hep3B cells. After the tumors reached 100 to 150 mm³ in size, mice received an intraperitoneal injection of YC-1 or a compound of Formula 1 (YC-1 mannose) (30 μg/g or 60 μg/g) or vehicle (DMSO) daily for 2 weeks. After the last treatment, the mice were euthanized, the tumors removed and analyzed. The results are shown in FIG. 16.

While the prsent invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process steps or steps, to the objective, spirit and scope of the present invention. All such modifications are intended to be within the scope of the claims appended hereto. All patents and publications cited above are hereby incorporated by reference.

1. A method of inhibiting HIF-1α expression in tumor cells or tissues, comprising contacting the tumor cells or tissues with a composition comprising 3-(5′-hydroxymethyl-2′-furyl)-1-benzylindazole at an effective amount for inhibiting HIF-1α expression, wherein said tumor is selected from the group consisting of hepatoma, stomach carcinoma, renal carcinoma, cervical carcinoma, neuroblastoma, and prostate carcinoma.

2. A method of inhibiting HIF-1-regulated gene expression in tumor cells or tissues, comprising contacting the tumor cells or tissues with a composition comprising 3-(5′-hydroxymethyl-2′-furyl)-1-benzylindazole at an effective amount for inhibiting HIF-1-regulated gene expression, wherein said tumor is selected from the group consisting of hepatoma, stomach carcinoma, renal carcinoma, cervical carcinoma, neuroblastoma, and prostate carcinoma.

3. A method of inhibiting angiogenesis in tumor cells or tissues, comprising contacting the tumor cells or tissues with a composition comprising 3-(5′-hydroxymethyl-2′-furyl)-1-benzylindazole at an effective amount for inhibiting angiogenesis, wherein said tumor is selected from the group consisting of hepatoma, stomach carcinoma, renal carcinoma, cervical carcinoma, neuroblastoma and prostate carcinoma.

4. A method of inhibiting tumor growth in animal tissues, comprising contacting the tumor cells or tissues with a composition comprising 3-(5′-hydroxymethyl-2′-furyl)-1-benzylindazole at an effective amount for inhibiting tumor growth, wherein said tumor is selected from the group consisting of hepatoma, stomach carcinoma, renal carcinoma, cervical carcinoma, neuroblastoma, and prostate carcinoma.

5. A method of inhibiting tumor progression and metastasis in animal tissues, comprising contacting the tumor cells or tissues with a composition comprising 3-(5′-hydroxymethyl-2′-furyl)-1-benzylindazole at an effective amount for inhibiting tumor progression and metastasis, wherein said tumor is selected from the group consisting of hepatoma, stomach carcinoma, renal carcinoma, cervical carcinoma, neuroblastoma, and prostate carcinoma.

6. A method of treating a HIF-1-mediated disorder or condition in a mammal comprising adminstering to the mammal a composition comprising a therapeutically effective amount of 3-(5′-hydroxymethyl-2′-furyl)-1-benzylindazole, wherein said HIF-1-mediated disorder or condition is selected from the group consisting of hepatoma, stomach carcinoma, renal carcinoma, cervical carcinoma, neuroblastoma, and prostate carcinoma.

7. A method of inhibiting HIF-1α expression in tumor cells or tissues, comprising contacting the tumor cells or tissues with a composition comprising a compound or mixture of compounds of the Formula I at an effective amount for inhibiting HIF-1α expression: 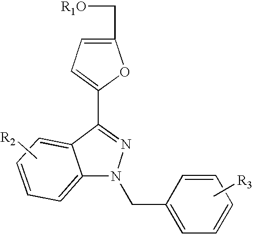
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
including single isomers, mixtures of isomers, and pharmaceutically acceptable solvates and salts thereof.

8. The method of claim 7, wherein said tumor is selected from the group consisting of hepatoma, stomach carcinoma, renal carcinoma, cervical carcinoma and neuroblastoma, and prostate carcinoma.

9. A method of inhibiting HIF-1-regulated gene expression in tumor cells or tissues, comprising contacting the tumor cells or tissues with a composition comprising a compound or mixture of compounds of the Formula I at an effective amount for inhibiting HIF-1-regulated gene expression: 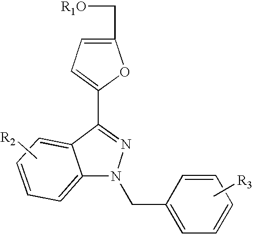
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
including single isomers, mixtures of isomers, and pharmaceutically acceptable solvates and salts thereof.

10. The method of claim 9 wherein said HIF-1-regulated gene is selected from the group consisting of erythropoietin, transferrin, transferrin receptor, ceruloplasmin, vascular endothelial growth factor (VEGF), VEGF receptor FLT-1, transforming growth factor β3, plasminogen activator inhibitor 1, α1B adrenergic receptor, adrenomedullin, endothelin 1, nitric oxide synthase 2, heme oxygenase 1, glucose transporter 1 and 3, hexokinase 1 and 2, enolase 1, glyceraldehyde-3-phosphate dehydrogenase, phosphoglycerate kinase 1, phosphoglucokinase L, pyruvate kinase M, aldolase A and C, rios phosphate isomerase, lactate dehydrogenase A, carbonic anhydrase 9, adenylate kinase 3, propyl-4-hydroxylase a1, insulin-like growth factor (IGF) 2, IGP-binding protein 1, 2 and 3, P21, Nip3, cyclin G2 and differentiated embryo chondrocyte 1.

11. The method of claim 10, wherein said HIF-1-regulated gene is selected from the group consisting of VEGF, aldolase A and enolase 1.

12. The method of claim 9, wherein said tumor is selected from the group consisting of hepatoma, stomach carcinoma, renal carcinoma, cervical carcinoma, neuroblastoma, and prostate carcinoma.

13. A method of inhibiting angiogenesis in tumor cells or tissues, comprising contacting the tumor cells or tissues with a composition comprising a compound or mixture of compounds of the Formula I at an effective amount for inhibiting angiogenesis: 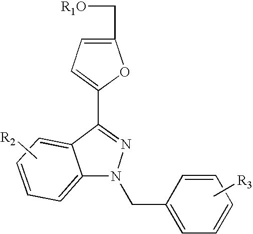
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
including single isomers, mixtures of isomers, and pharmaceutically acceptable solvates and salts thereof.

14. The method of claim 13, wherein said tumor is selected from the group consisting of hepatoma, stomach carcinoma, renal carcinoma, cervical carcinoma, neuroblastoma, and prostate carcinoma.

15. A method of inhibiting tumor growth in animal tissues, comprising contacting the tumor cells or tissues with a composition comprising a compound or mixture of compounds of the Formula I at an effective amount for inhibiting tumor growth: 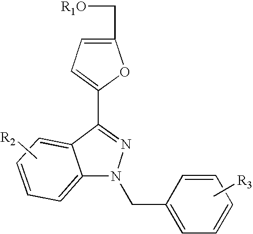
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
including single isomers, mixtures of isomers, and pharmaceutically acceptable solvates and salts thereof.

16. The method of claim 15, wherein said tumor is selected from the group consisting of hepatoma, stomach carcinoma, renal carcinoma, cervical carcinoma, neuroblastoma, and prostate carcinoma.

17. A method of inhibiting tumor progression and metastasis in animal tissues, comprising contacting the tumor cells or tissues with a composition comprising a compound or a mixture of compounds of the Formula I at an effective amount for inhibiting tumor progression and metastasis: 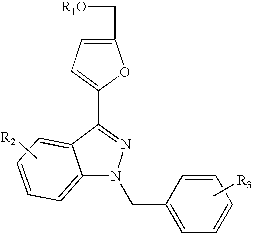
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
including single isomers, mixtures of isomers, and pharmaceutically acceptable solvates and salts thereof.

18. The method of claim 17, wherein said tumor is selected from the group consisting of hepatoma, stomach carcinoma, renal carcinoma, cervical carcinoma, neuroblastoma, and prostate carcinoma.

19. A method of treating a HIF-1-mediated disorder or condition in a mammal comprising administering to the mammal a composition comprising a therapeutically effective amount of a compound or a mixture of compounds of the Formula I: 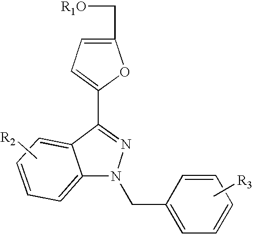
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
including single isomers, mixtures of isomers, and pharmaceutically acceptable solvates and salts thereof.

20. The method of claim 19, wherein said HIF-1-mediated disorder or condition is selected from the group consisting of hepatoma, stomach carcinoma, renal carcinoma, cervical carcinoma, neuroblastoma, and prostate carcinoma.

21. A pharmaceutical composition comprising an amount effective of a compound or mixture of compounds of the Formula I to inhibit tumor angiogenesis, tumor growth or tumor progression and metastasis in a host in need thereof, or to treat an HIF-1-mediated disorder or condition in a mammal; and a pharmaceutically acceptable carrier: 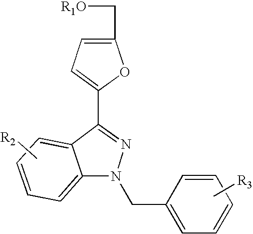
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
including single isomers, mixtures of isomers, and pharmaceutically acceptable solvates and salts thereof.

22. A compound of the Formula I: 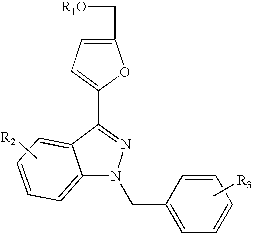
wherein:
R₁ is a polyol; and
R₂ and R₃ are independently chosen from hydrogen, optionally substituted alkyl, optionally substituted alkoxy, halogen, nitro, substituted amino, alkylsulfonyl, alkylsulfanyl, aminocarbonyl, alkoxycarbonyl, optionally substituted aryl and optionally substituted heteroaryl;
including single isomers, mixtures of isomers, and pharmaceutically acceptable solvates and salts thereof.

23. A compound according to claim 22 wherein R₂ and R₃ are hydrogen.

24. A compound according to claim 22 wherein R₁ is a 2-hydroxymethyl-pyran-3,4,5-triol-6-yl.

25. A compound according to claim 24 wherein R₂ and R₃ are hydrogen.
